(19) United States
(12) Patent Application Publication
(10) Pub. No.: US 2016/0136605 A1
(43) Pub. Date: May 19, 2016

(54) COLLECTOR PIPE FOR A RADIAL-BED REACTOR

(71) Applicant: IFP Energies Nouvelles, Rueil-Malmaison Cedex (FR)

(72) Inventors: Frederic BAZER-BACHI, Irigny (FR); Cecile PLAIS, Les Haies (FR); Mehdi LE MOEL, Oullins (FR); Eric SANCHEZ, Saint Genis Laval (FR)

(73) Assignee: IFP ENERGIES NOUVELLES, Rueil-Malmaison Cedex (FR)

(21) Appl. No.: 14/939,005

(22) Filed: Nov. 12, 2015

(30) Foreign Application Priority Data
Nov. 14, 2014 (FR) 14/61004

(51) Int. Cl. B01J 8/08; C07C 5/22; C07C 6/04; C10G 11/00

(52) U.S. Cl. CPC B01J 8/085; C10G 11/00; C07C 5/22; C07C 6/04; B01J 2208/00752; B01J 2208/00761; B01J 2208/00884; B01J 2208/00893

(57) ABSTRACT

This invention relates to a collector pipe (8) that is permeable to a gaseous fluid and able to retain particles having a dimension that is greater than a minimum dimension, in which the cross-section of said pipe is a convex polygon having at least three sides.

This invention relates to a collector pipe for moving-bed units having a radial circulation of the feedstock and of the reagents from the periphery of the reaction chamber toward the center. A person skilled in the art characterizes as “radial” a flow of gaseous reagents taking place through a generally mobile catalytic bed according to a set of directions corresponding to radii that are oriented from the periphery toward the center, or from the center of the chamber toward the periphery. This invention also has as its object a reactor having a radial bed comprising a pipe for collecting reaction effluent according to the invention. Finally, the invention relates to a method for catalytic conversion of a feedstock of hydrocarbons using a radial-bed reactor.

### STATE OF THE ART

The most representative unit of this type of radial flow is the regenerative reforming of hydrocarbon fractions of the gasoline type that can be defined as having a distillation interval of between 80 and 250° C. The field of application of this invention, however, is broader, and in addition to catalytic reforming of gasolines, there can be cited the skeletal isomerization of various C4, C5 olefinic fractions, or even the metathesis method for producing propylene, for example. This method list is not exhaustive, and this invention can be applied to any type of catalytic method having radial flow and gaseous feedstock. Thus, in the context of new energy technologies, the ethanol to diesel method, for example, could use this type of technology.

Some of these radial-bed units, including regenerative reforming, rely on a flow of the so-called moving-bed catalyst, i.e., a slow gravity flow of the confined catalyst particles (or catalytic bed) in an annular space limited by the wall of the reactor and an interior wall that is permeable to gas and impermeable to the catalyst grains, which corresponds to the collector pipe (or central collector) that recovers the reaction effluents. Alternatively, the catalytic moving bed can be confined in a generally annular space, formed between a so-called “external” grid and the collector pipe that are arranged preferably in a concentric manner. The so-called “external” grid can be formed by an assembly of grid elements in the shape of shells (shells in English terminology).

The gaseous feedstock is generally introduced through the external periphery of the annular bed and passes through the catalytic bed in a manner that is approximately perpendicular to the vertical direction of flow of the latter. The reaction effluents are then recovered in the collector pipe (or collector).

The use of this type of reactor is, however, limited in terms of feedstock flow. Actually, a feedstock flow that is too high will lead to a phenomenon of jamming of the catalyst against the central collector (or “jamming” according to English terminology). The force exerted by the feedstock circulating radially from the external periphery of the catalyst bed toward the center on the bed of catalyst grains presses them against the wall of the central collector, which increases the frictional stress that then resists the sliding of the grains along the wall. If the feedstock stream is high enough, then the friction force that results from it is enough to bear the weight of the catalytic bed so that the gravity flow of the catalyst grains ceases, at least in certain areas adjacent to the wall of the central collector. In these areas, the catalyst grains are then said to be “jammed” (“pinned” according to English terminology) by the gas flow and are kept immobile against the wall of the collector. The phenomenon of immobilization of the catalyst grains should be strongly avoided in reactors for catalytic reforming of hydrocarbon feedstock to the extent that it promotes the reactions of deactivation of the catalyst (for example by coking), thus preventing the continued exploitation of the reactor. Potentially, when the catalyst cake becomes too thick along the pipe, it is then necessary to reduce the flow of gas to be treated or even to stop the unit for the purpose of unclogging said pipe.

One purpose of the invention is therefore to propose a new type of pipe for collecting gaseous effluent for a radial-bed reactor whose design makes it possible to limit the phenomena of “jamming” of the catalyst grains. Thus, a reactor using the effluent collector according to the invention can be operated for longer periods and/or by using larger hydrocarbon feedstock flows (increasing the capacity of the unit).

### SUMMARY OF THE INVENTION

This invention relates to a pipe for collecting a gaseous fluid that is permeable to a gaseous fluid and able to retain particles having a dimension that is greater than a minimum dimension, in which the cross-section of said pipe is a convex polygon having at least three sides. In the context of the invention, by the term “impermeable to particles” is meant the fact that the openings through which the gaseous fluid diffuses are smaller than the minimum distance taken between two opposite points of the particle so as not to allow said particle to pass through the openings.

Surprisingly, the applicant has found that a collector pipe with a polygonal cross-section having at least three sides is less subject to the “jamming” phenomenon for the same feedstock flow compared with a collector pipe according to the prior art.

According to the invention, the sides of the polygon are straight or have a radius of curvature.

The section of the pipe can comprise between four and thirty sides, preferably between five and twenty sides. For example, the section can have a shape that is hexagonal (six sides) or octagonal (eight sides) or decagonal (ten sides) or even dodecagonal (twelve sides).

According to the invention, the polygon is uniform (the sides being of equal length) or non-uniform, preferably uniform.

The pipe according to the invention can be formed by an assembly of grids or perforated plates, for example by welding. When the pipe consists of grids, they can be a mesh of shaped metal rods and wires.

In a preferred embodiment, the pipe that is permeable to a gaseous fluid and impermeable to the particles appears in the form of a grid of the Johnson type.

This invention also has as its object a reactor having radial circulation of gaseous fluid comprising:
an external casing defining a chamber extending along a vertical main axis and containing a reaction zone comprising a bed of catalyst particles;
at least one input means of a feedstock;
at least one output means of the effluent produced by the catalytic reaction;
at least one input means of the catalyst to introduce the catalyst into the reaction zone;
at least one output means of the catalyst coming out from the reaction zone; and
a pipe for collecting effluent according to the invention, placed in the reaction zone, in communication with the output means of the effluent; and
optionally, a cylindrical grid that is permeable to gases and impermeable to catalyst particles, placed in the chamber in a manner that is concentric in relation to the collector pipe.

In a preferred embodiment of the reactor, the cylindrical grid is placed between the casing and the collector pipe so as to define an external annular zone between the casing and the cylindrical grid, an annular catalytic zone between the grid and the collector pipe, and a collector space delimited by the collector pipe.

The cylindrical grid can be an assembly of shell-shaped grid elements extending along the vertical axis in the reactor.

According to another embodiment of the reactor in which the cylindrical grid is not present, the reactor then comprises a plurality of tubes for distribution of the gaseous fluid, connected to a distribution box and immersed in the reaction zone.

Finally, the invention relates to a method for catalytic conversion of a gaseous hydrocarbon feedstock using a reactor according to the invention, in which:
the hydrocarbon feedstock is delivered continuously in gaseous form into the catalytic reaction zone contained in the reactor;
the hydrocarbon feedstock passing radially through the catalytic reaction zone is put into contact with the catalyst so as to produce a gaseous effluent; and
said effluent is drawn off after it passes through the collector pipe.

In a preferred manner, the method uses a catalytic moving bed so that the catalyst is delivered and drawn off continuously.

The catalytic conversion reaction is, for example, a catalytic reforming reaction, a skeletal isomerization of olefins, metathesis for the production of propylene, an oligomeric cracking reaction.

### DETAILED DESCRIPTION OF THE INVENTION

The other characteristics and advantages of the invention will become clear from reading the following description, given solely by way of illustration and nonlimiting, and to which are attached:

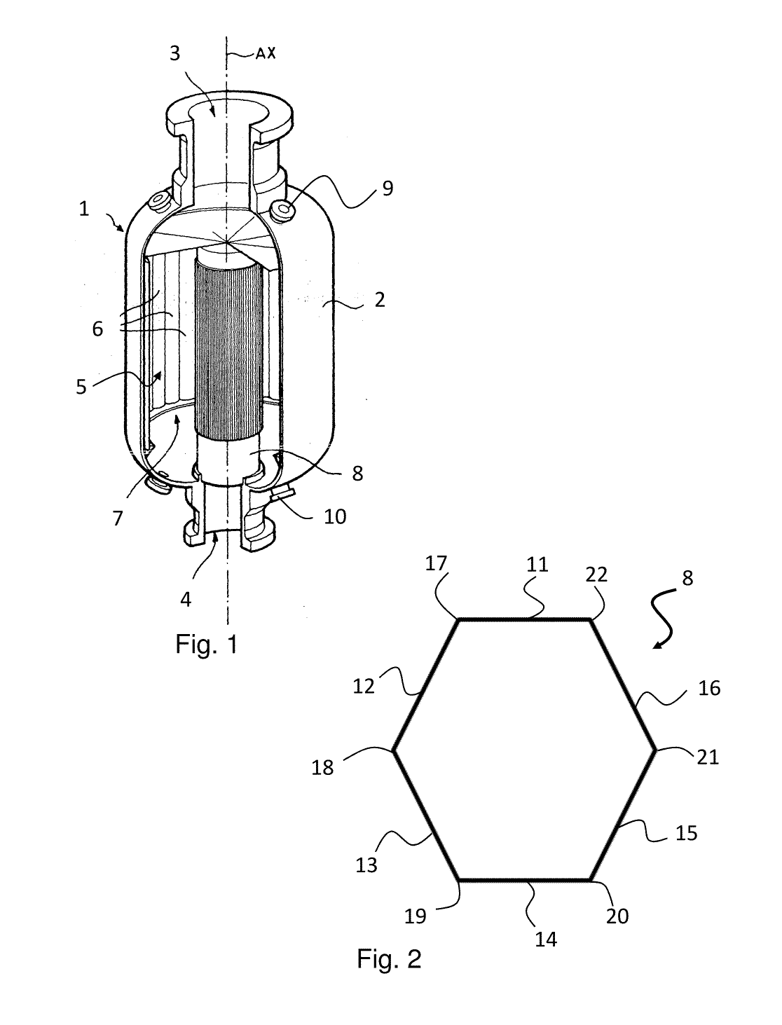

FIG. 1, which shows a perspective view with a partial view of a radial-flow reactor according to the prior art;

FIG. 2, which is a cutaway view perpendicular to the main axis of a pipe according to the invention;

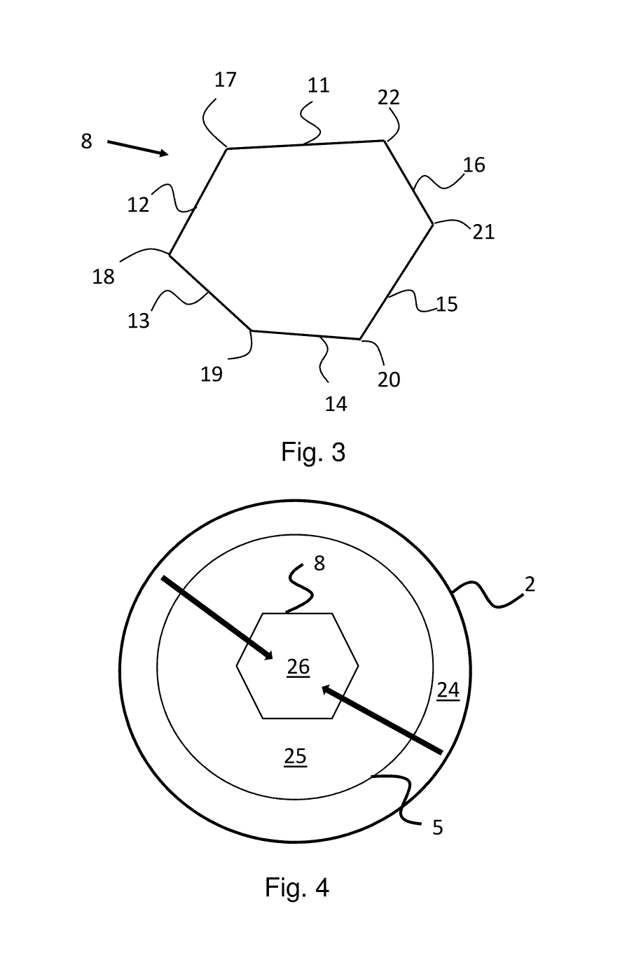

FIG. 3, which is a cutaway view perpendicular to the main axis of a pipe according to the invention according to another embodiment;

FIG. 4 is a cutaway view of a reactor according to the invention along a plane that is perpendicular relative to the main axis of the reactor;

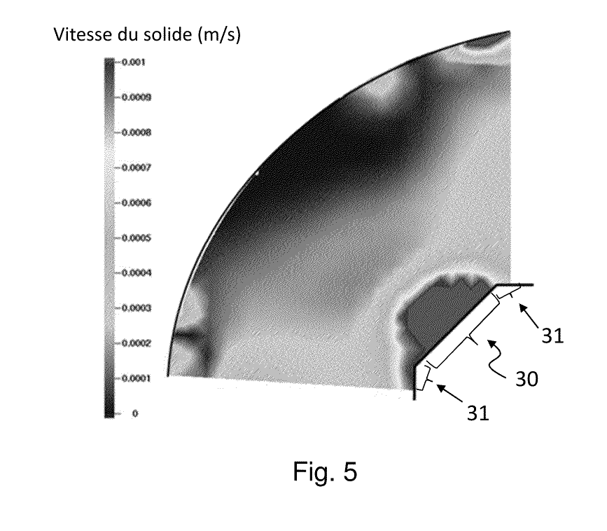

FIG. 5 is a graph illustrating the distribution of the speed of the solids in a portion of the annular zone of the catalytic bed between an external grid and the central collector.

With reference to FIG. 1, a radial-flow reactor 1 according to the prior art appears outwardly in the form of a carboy forming a cylindrical chamber 2 that extends along an axis of symmetry AX. In its upper part, the chamber 2 comprises a first opening 3, and in its lower part, a second opening 4. The openings 3 and 4 are intended respectively for the input and the output of a fluid passing through the reactor 1.

Inside this cylindrical tank is arranged a catalytic bed 7 having the shape of a vertical cylindrical ring limited on the interior side by a central cylindrical tube 8 formed by a so-called “internal” grid retaining the catalyst and on the exterior side by another so-called “external” grid 5 either of the same type as the internal grid or by a device consisting of an assembly of shell-shaped grid elements 6 extending longitudinally, as shown in FIG. 1. These shell-shaped grid elements 6 forming pipes are also known under the English term “shells.” These pipes 6 are held by the tank and flattened against the internal face of the chamber, parallel to the axis AX, to form an approximately cylindrical internal casing. The shell-shaped grid elements 6 are in direct communication with the first opening 3 via their upper end to receive a gaseous flow of feedstock. The gaseous flow spreads through the perforated wall of the pipes 6 to pass through the bed of catalytic solid particles 7 by converging radially toward the center of the reactor 1. The feedstock is thus put in contact with the catalyst so as to undergo chemical transformations, for example a catalytic reforming reaction, and to produce an effluent from the reaction. The effluent from the reaction is then collected by the central cylinder tube 8 (or collector pipe) extending along the axis AX and also having a perforated wall. This central cylinder 8 (or collector pipe) here is in communication with the second opening 4 of the reactor via its lower end.

During operation, the gaseous fluid introduced into the first opening 3 radially passes through the “external” grid 5, and then radially passes through the catalyst particle bed 7 where it is put in contact with the catalyst so as to produce an effluent that is eventually collected by the central cylinder 8 and evacuated through the second opening 4.

Such a reactor can also operate with a continuous gravity flow of catalyst in the annular catalytic bed 7. In the case of FIG. 1, the reactor 1 further comprises means for introducing the catalyst 9 into the annular bed, placed in an upper part of the reactor, and means for drawing off the catalyst 10 that are arranged in a lower part of the reactor.

Alternatively, as a means for distribution of the gaseous fluid in replacement of the external grid, the reactor can use a plurality of fluid distribution tubes, directly immersed in the catalytic bed and the tubes being connected to a distribution box. These distribution tubes, closed at their distal end, for example with a circular cross-section, are formed by a grid or a perforated plate that is permeable to gas and impermeable to catalyst grains.

So as to reduce the amount of catalyst immobilized along the wall of the collector pipe 8, a new concept of collector pipe is proposed that is different from that of FIG. 1. According to the invention, the collector pipe 8 thus comprises a cross-section (along a plane perpendicular to the central axis of the pipe) of a polygonal shape with at least three sides. The cross-section of the pipe forms a convex polygon, i.e., any straight segment joining two non-consecutive vertices of the polygon is contained, with the exception of its ends, in the interior of the polygon. According to the invention, the polygon can be uniform or non-uniform.

According to the invention, the cross-section of the pipe can comprise between four and thirty sides, preferably between five and twenty sides. For example, the cross-section can have a hexagonal or octagonal or decagonal shape.

With reference to FIG. 2, which is a cutaway view along a plane perpendicular to the central axis of the pipe, it is observed that the pipe 8 according to the invention comprises here six sides 11, 12, 13, 14, 15, 16 and six edges 17, 18, 19, 20, 21, 22. Furthermore, the six sides of the polygon have the same length 11, 12, 13, 14, 15, 16, thus forming a so-called uniform convex polygon. According to another embodiment shown in FIG. 3, the pipe comprises a cross-section with a convex hexagonal shape and whose lengths of the sides are different.

The collector pipe according to the invention is perforated so as to allow the gaseous fluid to pass through and to retain the catalyst solid particles whose dimensions are greater than a defined value. For example, the collector pipe can be made by the assembling and welding of plates comprising openings, preferably uniformly distributed on the surface of the pipe. Alternatively, the perforated plates are replaced by a grid obtained by a mesh of shaped metal rods and wires, for example a grid of the Johnson type.

The openings formed in the pipe are determined so as to retain catalyst particles that can take the form of a sphere or stick. For example, the pipe is configured to retain catalyst particles whose dimension is between 0.5 mm and 15 mm and often between 1 mm and 10 mm.

Surprisingly, the applicant observed that during its use in a radial reactor, the pipe according to the invention has zones, in the area of the edges, where the thickness of the immobilized catalyst cake is smaller. Actually, it has been found that in the area of the edges, the solid is less pressed against the wall of the pipe with the consequence that the cake can no longer expand from these edges.

Thus, by reducing the amount of catalyst “jammed” by the cake, the “inactive” catalyst portion is reduced, and therefore the catalytic performance of the reactor is improved.

The invention also relates to a method for catalytic treatment of a hydrocarbon feedstock in a radial reactor having a catalyst moving bed. The reactor comprises:
an external casing defining a chamber extending along a vertical main axis and containing a reaction zone having a bed of catalyst particles,
at least one input means of a feedstock,
at least one output means of the effluent produced by the catalytic reaction,
at least one input means of the catalyst for introducing the catalyst into the reaction zone;
at least one output means of the catalyst coming out from the reaction zone; and
optionally, a grid that is permeable to gases and impermeable to catalyst particles, which is placed in the chamber in a manner that is concentric in relation to the collector pipe.

The reactor includes, moreover, a pipe for collecting the effluent according to the invention that is placed in the reaction zone in communication with the output means of the effluent.

According to a preferred embodiment of the reactor (having centripetal radial circulation, i.e., the gaseous flow circulates from the periphery of the chamber toward the center of the chamber) shown in FIG. 4, the porous cylindrical grid 5 that is permeable to gases and impermeable to the catalyst is placed between the external casing 2 and the collector pipe 8 in a manner that is concentric in relation to the collector pipe. In such a configuration, the reactor comprises an “external” annular zone 24 between the casing 2 and the so-called “external” grid 5, an annular catalytic zone 25 between the so-called “external” grid 5 and the collector pipe 8 and a collector space 26 delimited by the collector pipe 8. The so-called “external” grid 5 can take the form of a perforated plate or of a grid formed by a mesh of shaped metal rods and wires or else an assembly of grid elements in the shape of a shell (or “shell” according to English terminology). During operation, the gaseous feedstock is injected either through the bottom or through the top of the reactor into the annular distribution zone 24, then passes through the so-called “external” grid 5 and next passes in an approximately radial manner through the bed of catalyst particles of the annular catalytic zone 25. In the annular catalytic zone 25, the gaseous fluid is put in contact with the catalyst to produce a reaction effluent, generally gaseous, which is collected in the space 26 of the collector pipe 8 and which is then drawn off either at the top of the reactor (when the feedstock is introduced at the bottom of the reactor) or at the bottom of the reactor (when the feedstock is introduced through the top of the reactor).

According to an alternative embodiment (not shown), the reactor does not have a cylindrical grid 5 but a plurality of distribution tubes, connected to a distribution box and immersed in the reaction zone, which make it possible to distribute the gaseous feedstock in the reaction zone.

According to an embodiment of a reactor according to the invention (not shown), the collector pipe 8 is arranged between the chamber 2 and the cylindrical grid 5 and in a way that is concentric in relation to the grid 5. In this arrangement, the collector 8 is equivalent to an “external” grid, and the grid 5 corresponds to an “internal” grid. In other words, the reactor comprises an “exterior” collector zone between the casing 2 and the collector pipe 8, an essentially annular catalytic zone delimited by the so-called “internal” grid 5 and the collector pipe 8 and a space for distribution of the gaseous fluid delimited by the cylindrical grid 5. During operation, the gaseous feedstock is introduced either through the top or through the bottom of the reactor via the pipe formed by the cylindrical grid 5 (the grid being closed at the end opposite the end in communication with the input means of the feedstock). The feedstock spreads through the grid 5 and passes in an approximately radial manner through the catalytic reaction zone where it is put in contact with the catalyst. A reaction effluent is collected in the external collector zone after having spread through the collector pipe 8 and that is then drawn off either at the top of the reactor (when the feedstock is introduced at the bottom of the reactor) or at the bottom of the reactor (when the feedstock is introduced through the top of the reactor).

It should be noted that the reactor according to the invention can be a reactor having a catalytic moving bed, i.e., the catalyst is introduced into the reactor and drawn off from said reactor continuously.

The reactor and the method according to the invention can be applied to perform reactions having radial circulation of gaseous fluid, such as, for example, a reaction for catalytic reforming of a hydrocarbon fraction, a skeletal isomerization of olefins, metathesis for the production of propylene, an oligomeric cracking reaction.

### Example

The movements of the gas and of the solid have been simulated in a mobile radial reactor with the following characteristics:
diameter of the external grid: 2.4 m
diameter of the central collector: 0.9 m
input gas velocity: 0.3 cm/s
flow of solid: 0.2 kg/s
load density of the catalyst: 700 kg/m³
catalyst diameter: 2 mm
density of the gas: 1.7 kg/m³
viscosity of the gas: 2.10⁻⁵ Pa·s

The central collector has been modeled in a uniform octagonal configuration in which the octagon circumscribes a circle with a diameter approximately equal to that of the central collector with a circular cross-section.

FIG. 5 is the result of a digital simulation (Computational Fluid Dynamics) obtained by means of the Barracuda® software (published by CPFD Software LLC) making it possible to measure the flow velocities of the solid in the reactor in the centripetal direction, i.e., the one that follows the gaseous flow (from the exterior to the interior of the reactor).

The results of the simulation show an acceleration of the velocity of the solid near the central collector. This velocity is maximum in the area of a region around the center of the side of the octagon (zone 30 in FIG. 5). Nevertheless, the presence of zones is found (zone 31 in FIG. 5) near the edges, where the velocity is clearly lower.

Now, it is known that the greater the velocity of the horizontal flow of reagents through the catalytic bed, the more it creates friction forces between the particles and the wall of the collector. Starting at a certain velocity, the friction forces are enough to counterbalance the gravitational force being exerted on the particles so that they find themselves pressed and then immobilized against the wall of the collector.

As a result of the presence of edges, the velocity of the particles is limited, which has the consequence of limiting the “jamming” phenomenon in the area of the zones adjacent to the edges so that the cake that forms on a side of the polygon cannot grow. Thus, if in the area of certain zones of the pipe, this horizontal fluid velocity becomes practically zero, the solid is no longer trapped by the gas flow and can flow along the pipe so that the cake that has formed between two edges can no longer expand.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

The entire disclosures of all applications, patents and publications, cited herein and of corresponding French Application No. 14/61004, filed Nov. 14, 2014 are incorporated by reference herein.

1. Reactor having centripetal radial circulation of gaseous fluid comprising:
an external casing (2) defining a chamber extending along a vertical main axis and containing a reaction zone having a bed of catalyst particles;
at least one input means of a feedstock placed at the top of the reactor or at the bottom of the reactor;
at least one output means of the gaseous effluent produced by the catalytic reaction placed at the bottom of the reactor when the input means of the feedstock is placed at the top of the reactor or at the top of the reactor when the input means of the feedstock [is] placed at the bottom of the reactor;
at least one input means of the catalyst to introduce the catalyst into the reaction zone;
at least one output means of the catalyst coming out from the reaction zone; and
a collector pipe (8) placed in the reaction zone, which is permeable to the gaseous effluent and able to retain catalyst particles having a dimension greater than a minimum dimension, in which the cross-section of said pipe is a convex polygon having at least three sides, the pipe being in communication with the output means of the effluent.

2. Reactor according to claim 1, in which a cylindrical grid that is permeable to gases and impermeable to catalyst particles is placed between the casing (2) and the collector pipe so as to define an external annular zone (24) between the casing (2) and the cylindrical grid (5), an annular catalytic zone (25) between the grid (5) and the collector pipe (8), and a collector space (26) delimited by the collector pipe (8).

3. Reactor according to claim 2, in which the cylindrical grid (5) is an assembly of shell-shaped grid elements (6) extending along the vertical axis.

4. Reactor according to claim 1, in which said reactor comprises a plurality of tubes for distribution of the gaseous fluid that are connected to a distribution box and immersed in the reaction zone.

5. Reactor according to claim 1, in which the sides of the polygon of the pipe are straight or have a radius of curvature.

6. Reactor according to claim 1, in which the cross-section of the pipe comprises between four and thirty sides, preferably between five and twenty sides.

7. Reactor according to claim 1, in which the polygon is uniform or non-uniform, preferably uniform.

8. Reactor according to claim 1, in which the pipe is formed by an assembly of grids or of perforated plates.

9. Method for catalytic conversion of a hydrocarbon feedstock using a reactor according to claim 1, in which:
the hydrocarbon feedstock is delivered continuously in gaseous form into a catalytic bed contained in the reactor;
the hydrocarbon feedstock passing radially through the catalytic bed is put in contact with the catalyst so as to produce a gaseous effluent; and
said effluent is drawn off after it passes through the collector pipe.

10. Method according to claim 9, in which the catalytic bed is mobile, and the catalyst is continuously delivered and drawn off.

11. Method according to claim 9, in which the catalytic conversion is selected from a catalytic reforming reaction, a skeletal isomerization of olefins, metathesis for the production of propylene, an oligomeric cracking reaction.
